(19) United States
(12) Patent Application Publication
(10) Pub. No.: US 2017/0312457 A1
(43) Pub. Date: Nov. 2, 2017

(54) MOBILE IMAGING MODALITY FOR MEDICAL DEVICES

(71) Applicant: Nuance Designs of CT, LLC, Woodbridge, CT (US)

(72) Inventors: David DeSalvo, Lake Hiawatha, NJ (US); David Markham, Mitcheldean (GB)

(73) Assignee: Nuance Designs of CT, LLC, Woodbridge, CT (US)

(21) Appl. No.: 15/581,662

(22) Filed: Apr. 28, 2017

Related U.S. Application Data
(60) Provisional application No. 62/330,587, filed on May 2, 2016.
(60) Provisional application No. 62/400,349, filed on Sep. 27, 2016.

(51) Int. Cl. A61M 5/50; G06K 9/22; G09B 5/02; A61M 5/31; A61M 5/20

(52) U.S. Cl. CPC A61M 5/5086; A61M 5/3129; A61M 5/20; G09B 5/02; G06K 9/228; A61M 2205/502; A61M 2205/3584; A61M 2205/52; A61M 2205/6063; A61M 2205/6072; A61M 2205/6081; A61M 2205/6009

(57) ABSTRACT

A drug delivery device monitoring system is provided. The system includes a drug delivery device having a visually-identifiable feature reflecting a state or an indicia of the drug delivery device; an electronic recordation device configured to capture an image of the visually-identifiable feature and generate image data therefrom; and a computing system operable to perform image analysis on the image data to generate interpreted data therefrom. The interpreted data is provided to a stakeholder monitoring the drug delivery device.

### CROSS-REFERENCE TO RELATED APPLICATION

This application claims the benefit of U.S. Provisional Application No. 62/330,587 filed on May 2, 2016 and entitled “Mobile Imaging Modality for Drug Delivery Devices,” and U.S. Provisional Application No. 62/400,349 filed on Sep. 27, 2016 and entitled “Mobile Imaging Modality for Drug Delivery Devices,” the entire contents of which are expressly incorporated herein by reference.

### FIELD OF INVENTION

Embodiments of the present invention are related to monitoring systems for medical devices.

### BACKGROUND OF THE INVENTION

With the advent of smart technology, some medical devices are able to connect with other electronic devices and send information via a wireless signal, such as Bluetooth®, wifi, near field communication (NFC), or the like. The connection requires the medical device and other electronic device to be directly paired together or indirectly connected via a wireless network. Likewise, the additional components required to send electronic signals add cost and complexity to the production of the medical device as well as extra regulatory burdens. There exists a need for a drug delivery device monitoring system that does not suffer from these disadvantages.

### SUMMARY OF THE INVENTION

In accordance with one aspect of the present invention, a drug delivery device monitoring system is provided. The system includes a drug delivery device having a visually-identifiable feature reflecting a state or an indicia of the drug delivery device; an electronic recordation device configured to capture an image of the visually-identifiable feature and generate image data therefrom; and a computing system operable to perform image analysis on the image data to generate interpreted data therefrom. The interpreted data is then provided to a stakeholder monitoring the drug delivery device.

In accordance with another aspect of the present invention, a software application executable on a mobile device is provided. The software application is provided to a user for monitoring the use of a medical device, such as a drug delivery device. The application provides the user with various options, including options for capturing an image of a visually-identifiable feature reflecting a state or an indicia of the drug delivery device. The application processes the image to produce image data, which is then further processed to extract and record the state and/or indicia.

### BRIEF DESCRIPTION OF THE DRAWINGS

FIG. 1 depicts an exemplary autoinjector in accordance with the present invention.

FIGS. 2a and 2b depict an exemplary autoinjector with graduation lines for measuring an amount of medicament, in accordance with the present invention.

FIGS. 3a through 3c depict an exemplary autoinjector with indicator text for measuring an amount of medicament, in accordance with the present invention.

FIGS. 4a and 4b depict an exemplary autoinjector with color markings for measuring an amount of medicament, in accordance with the present invention.

FIGS. 5a and 5b depict an exemplary autoinjector with text markings for determining whether the autoinjector is new or used, in accordance with the present invention.

FIG. 6 depicts an exemplary autoinjector with a transparent driving region, in accordance with the present invention.

FIG. 7 depicts a medical device monitoring system, in accordance with the present invention.

FIG. 8 depicts an exemplary computing system for use in a medical device monitoring system, in accordance with the present invention.

FIG. 9 depicts an exemplary computing device for use in a medical device monitoring system, in accordance with the present invention.

FIG. 10 is a flow chart depicting a process for administering a medicament and monitoring a medical device, in accordance with the present invention.

FIG. 11 is a flow chart depicting another process for administering a medicament and monitoring a medical device, in accordance with the present invention.

FIG. 12a is an Opening Screen of a mobile application, in accordance with the present invention.

FIG. 12b is a Passcode Screen of a mobile application, in accordance with the present invention.

FIG. 12c is a Main Menu Screen of a mobile application, in accordance with the present invention.

FIG. 12d is a Dashboard Screen of a mobile application, in accordance with the present invention.

FIG. 12e is a Training Status Screen of a mobile application, in accordance with the present invention.

FIG. 12f is a Scanning Screen of a mobile application, in accordance with the present invention.

FIG. 12g is a Scanning Results Screen of a mobile application, in accordance with the present invention.

FIG. 12h is an Injection Site Screen of a mobile application, in accordance with the present invention.

FIG. 12i is a Troubleshooting Screen of a mobile application, in accordance with the present invention.

FIG. 12j is a Data Record Screen of a mobile application, in accordance with the present invention.

FIG. 12k is a Training Screen of a mobile application, in accordance with the present invention.

FIG. 12l is a Training Screen of a mobile application with a Materials sub-menu selected, in accordance with the present invention.

FIG. 12m is a Training Screen of a mobile application with a Tests sub-menu selected, in accordance with the present invention.

FIG. 12n is a Social Screen of a mobile application, in accordance with the present invention.

FIG. 12o is a Social Screen of a mobile application with a Messages sub-menu selected, in accordance with the present invention.

FIG. 12p is a Social Screen of a mobile application with a Friends sub-menu selected, in accordance with the present invention.

FIG. 13a depicts a barcode, in accordance with the present invention.

FIG. 13b depicts a QR code, in accordance with the present invention.

FIG. 13c depicts a light-emitting diode, in accordance with the present invention.

FIG. 13d depicts a holographic print, in accordance with the present invention.

FIG. 13e depicts a microprint, in accordance with the present invention.

FIG. 13f depicts a watermark, in accordance with the present invention.

### DETAILED DESCRIPTION

Reference will now be made in detail to the preferred embodiments of the invention illustrated in the accompanying drawings. Wherever possible, the same or like reference numbers will be used throughout the drawings to refer to the same or like features. It should be noted that the drawings are in simplified form and are not drawn to precise scale. In reference to the disclosure herein, for purposes of convenience and clarity only, directional terms such as top, bottom, above, below and diagonal, are used with respect to the accompanying drawings. Such directional terms used in conjunction with the following description of the drawings should not be construed to limit the scope of the invention in any manner not explicitly set forth. Additionally, the term “a,” as used in the specification, means “at least one.” The terminology includes the words above specifically mentioned, derivatives thereof, and words of similar import. “About” as used herein when referring to a measurable value such as an amount, a temporal duration, and the like, is meant to encompass variations of ±20%, ±10%, ±5%, +1%, and +0.1% from the specified value, as such variations are appropriate.

Ranges throughout this disclosure and various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

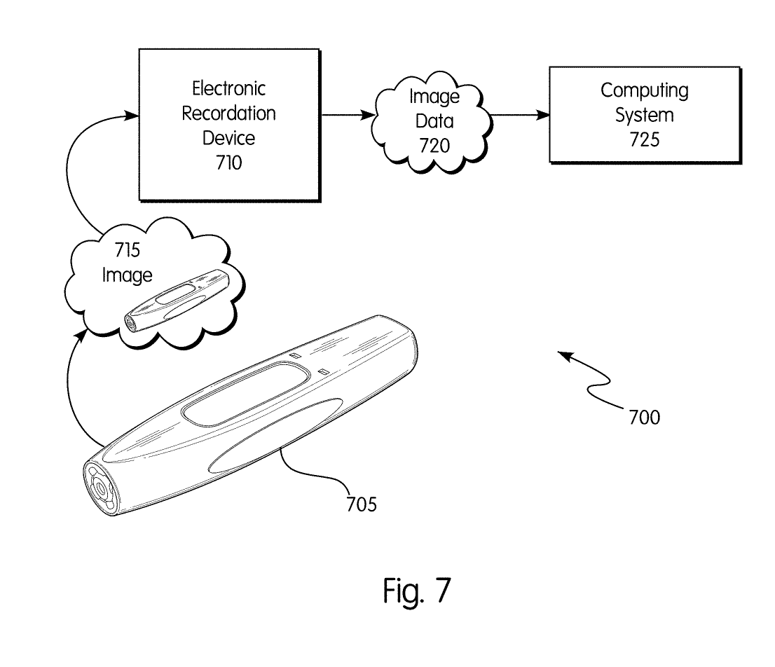

Referring now to FIG. 7, there is seen an exemplary medical device monitoring system 700 in accordance with the present invention. Monitoring system 700 includes a medical device 705 to be monitored, an electronic recordation device 710 for obtaining an image 715 associated with a state or indicia of medical device 705 and producing image data 720 therefrom, and a computing system 725 for processing the image data into interpreted data to be provided to a stakeholder.

Medical device 705 may include any medical equipment or other apparatus to be monitored. For example, medical device 705 may include a drug delivery device, such as an autoinjector (e.g., a pen-injector or other wearable injector), syringe, nasal spray, EpiPen®, infusion pump, IV drip, a wearable injector, or any other personal dispensing device, such as one for dispensing medicines and/or fluids. In one embodiment, medical device 705 includes an autoinjector configured to automatically inject a dose of medicament when actuated.

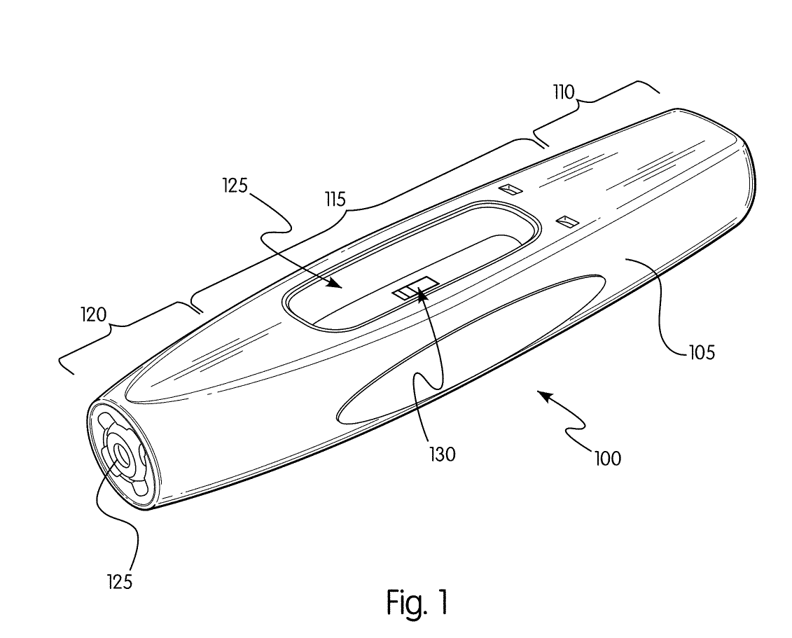

Referring now to FIG. 1, there is seen an exemplary autoinjector 100 in accordance with the present invention. Autoinjector 100 includes components configured to inject within a user a measured dose of a medicament stored within a syringe positioned inside autoinjector 100. For this purpose, autoinjector 100 includes a body 105 having an actuation region 110, a driving region 115, a needle shield 120, and a viewing area 125 positioned on body 105 (e.g., a side of body 105) for viewing at least one visually-identifiable feature 130 of autoinjector 100. To inject the medicament, the user positions needle shield 120 of autoinjector 100 at an injection site against his/her skin. Depressing actuation region 110 causes insertion of a needle through needle shield 120 at end 125 and into the skin of the user. The measured dose of medicament is then automatically injected into the user through the needle.

The body of autoinjector 100 may be constructed as a unitary piece or from multiple pieces, and may be manufactured (such as via casting or 3D printing) or handcrafted from any material(s) of sufficient strength and stiffness to enable autoinjector 100 to operate as intended, such as metal (e.g., titanium, precious metals), silicone, plastic, resin, composites, rigid 3D printed materials, non-corrosive materials, stiff hypoallergenic materials, etc.

Visually-identifiable feature 130 may be positioned within viewing area 125 and/or at other locations on autoinjector 100, and may include, for example, any visual feature indicative of a state of autoinjector 100 (e.g., a property of autoinjector 100 that can change, such as over time or after an event). For example, visually-identifiable feature 130 may indicate the time autoinjector 100 was last used and/or a volume of medicament remaining within autoinjector 100. Visually-identifiable feature 130 may also indicate whether autoinjector 100 is expired/unexpired, empty/full of medicament (e.g., when the medicament is visible through a transparent viewing area 125), new/used, properly/improperly used, intact, damaged, tampered, or combinations thereof.

Visually-identifiable feature 130 may also include any visual feature indicative of an indicia of autoinjector 100 (e.g., a property of autoinjector 100 that is permanent or changes only upon re-loading the autoinjector). For example, visually-identifiable feature 130 may include a production lot associated with autoinjector 100 or the medicament, serialized information of the individual autoinjector, an expiration date, instructions for use, a prescribed time of use, patient identifying information, prescription information, information linked to a support group, or combinations thereof. Visually-identifiable feature 130 may also include combinations of any number of state and/or indicia features.

Autoinjector 100 may include a plurality of visually-identifiable features 130, including features indicative of both a state and an indicia of autoinjector 100. For example, autoinjector 100 may be provided with one visually-identifiable feature 130 indicative of the volume of medicament currently within autoinjector 100 and another visually-identifiable feature 130 in the form of text communicating an expiration date of the medicament. Alternatively, the state and indicia may be combined into a single visually-identifiable feature 130. For example, the visually-identifiable feature 130 may be formed as a mark that appears only after autoinjector 100 has been used (state), the mark being, for example, a barcode representing patient identifying information or information about the medicament contained within autoinjector 100 (indicia).

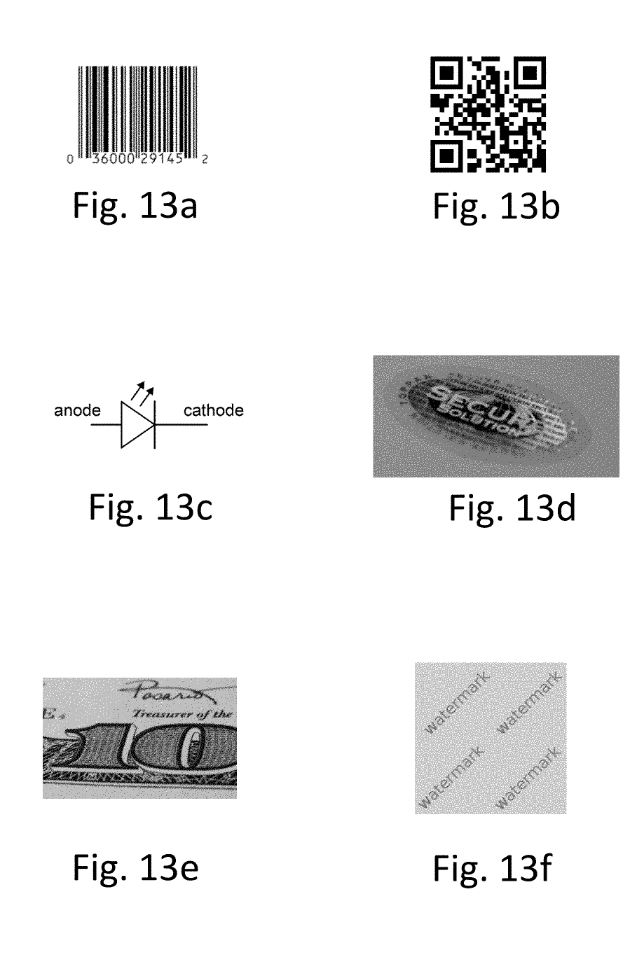

Whether indicating a state or indicia of autoinjector 100, visually-identifiable feature 130 may be formed from any of various types of externally viewable markings, marking materials, and security features positioned within viewing area 125 and/or about various other locations on body 105 of autoinjector 100. Visually-identifiable feature 130 may include, for example, the position of a plunger tip with respect to a syringe, a barcode (see FIG. 13a), a QR code (see FIG. 13b), a graduation line, a light emitting diode (LED) (see FIG. 13c), printed text, a holographic print (see FIG. 13d), a microprint (see FIG. 13e), color-shifting ink, a watermark (see FIG. 13f), an appearing mark, a disappearing mark, or combinations thereof. Visually-identifiable feature 130 may also include markings that are covert and/or invisible to the naked eye, such as markings created using infrared or ultraviolet ink. Such covert and/or invisible markings may be useful, for example, to track autoinjector 100, verify authenticity of autoinjector 100 or its medicament, prevent counterfeiting of autoinjector 100, or prevent theft thereof.

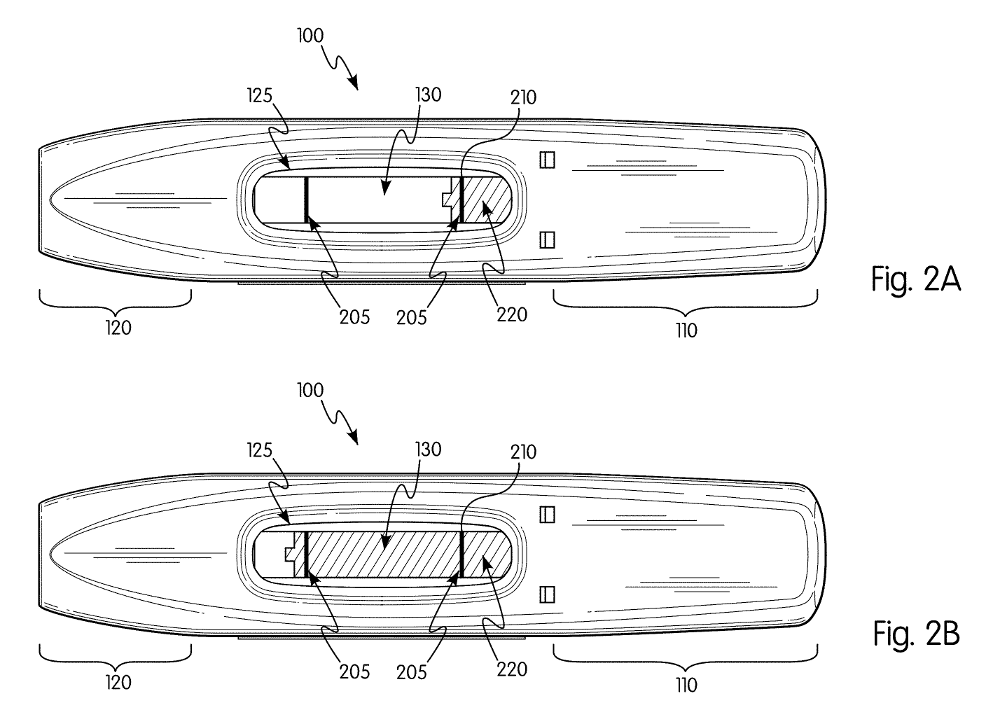

Referring now to FIGS. 2a and 2b, there is seen autoinjector 100 having a visually-identifiable feature 130 that includes a transparent syringe 210 having one or more graduation lines 205 associated with respective volume levels of a medicament 215. Depressing actuation region 110 causes a plunger 220 to advance within syringe 210 for administering a measured dose of medicament. The position of plunger 220 with respect to graduation lines 205 may be used to determine an amount of medicament remaining within syringe 210 (e.g., 1 mL in FIGS. 2a and 0.5 mL in FIG. 2b). This information may be used, for example, to determine whether syringe 210 includes enough medicament for a subsequent injection. Comparison of the amount of medicament both before (which is known in the event autoinjector 100 is new and unused) and after an injection may also be used to determine whether the injection dispensed the proper dose of medicament.

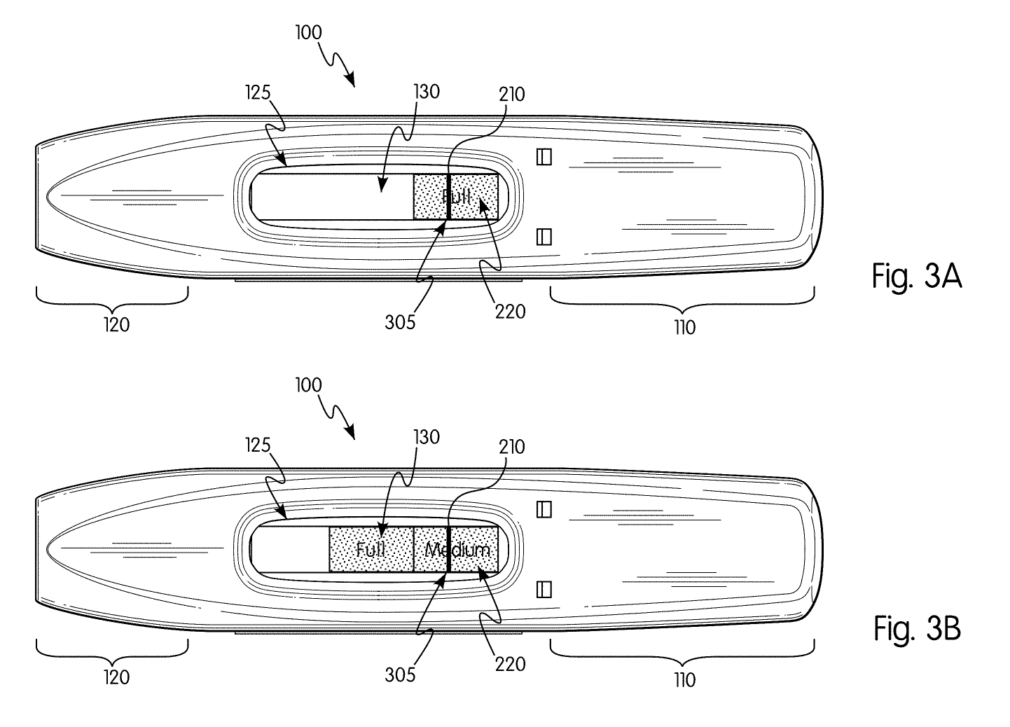

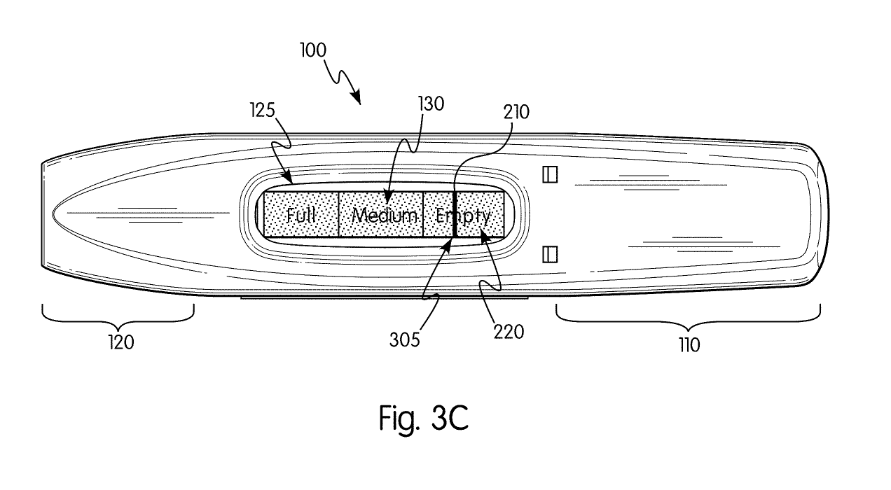

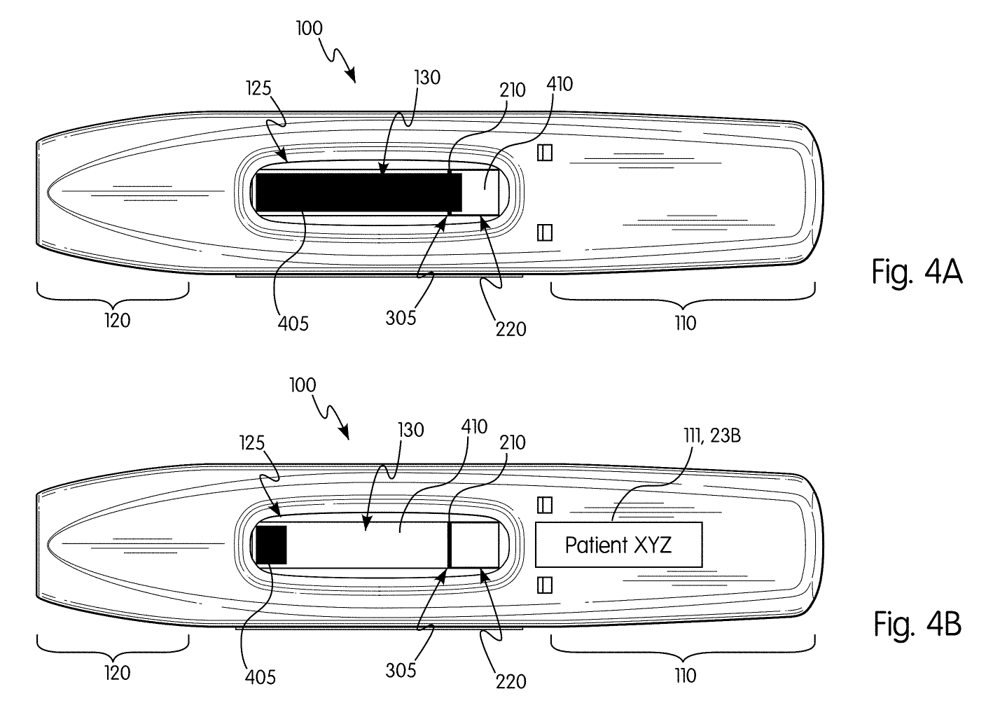

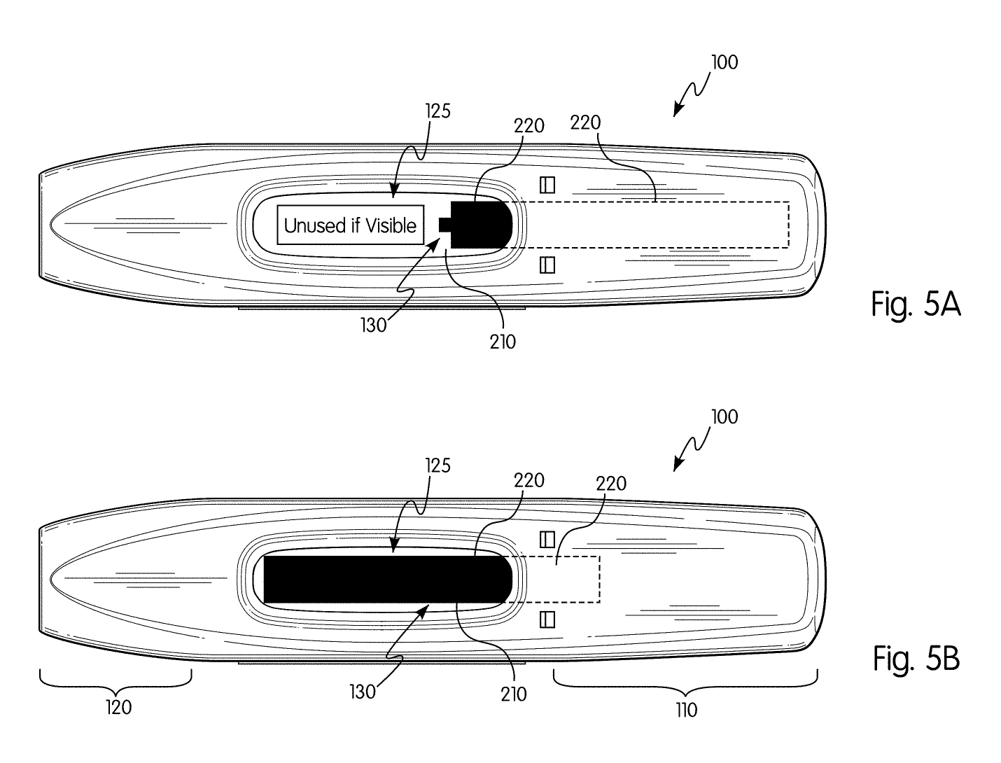

In an alternative embodiment, various portions of plunger 220 may be provided with text and/or different colors indicative of the amount of medicament remaining within syringe 210. For example, with respect to the embodiment depicted in FIGS. 3a through 3c, plunger 220 is provided with three different markings “Full,” “Medium,” and “Low.” As successive injections advance plunger 220 within syringe 210, marker 305 on syringe 210 indicates the state of the medicament at any given time. This information may be used, for example, to alert the user or prescribing physician of the current amount of medicament remaining within autoinjector 100 and/or to determine when to prescribe refills of the medicament. In alternative embodiments, such as the one depicted in FIGS. 4a and 4b, plunger 220 is provided with one or more colors indicative of an amount of medicament remaining, such that a first color (e.g., black 405 as in FIG. 4a) is visible when plunger 220 is retracted and other colors (e.g., gray 410 as in FIG. 4B) are visible when it is extended. In yet another embodiment, such as the one depicted in FIGS. 5a and 5b, a side of syringe 210 or an inner side of autoinjector 100 is provided with text, such as “used if visible.” This text is then covered or hidden as plunger 220 is advanced within syringe 210 to dispense the medicament. In another embodiment, text, such as “used,” is also provided on plunger 220, which text becomes visible within viewing area 125 as plunger 220 is advanced through syringe 210.

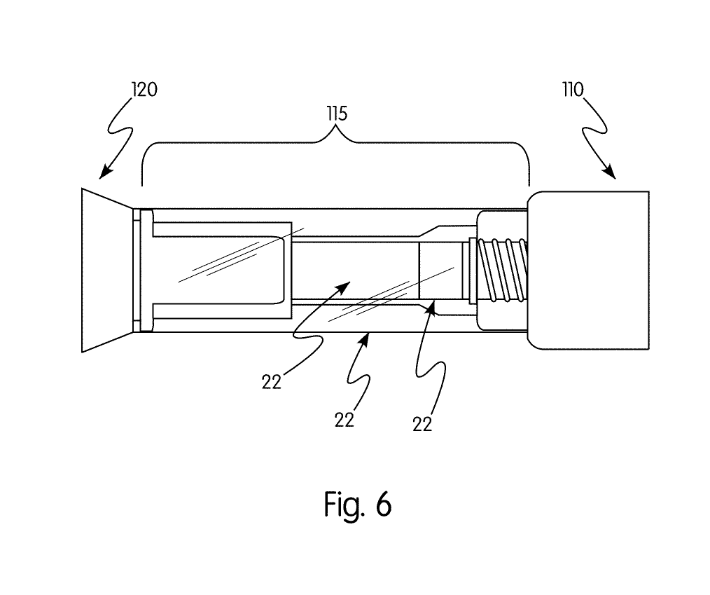

In still another exemplary embodiment, such as the one shown in FIG. 6, and in addition to or in lieu of viewing area 125, one or more portions of autoinjector 100 (e.g., actuation region 110, driving region 115, needle shield 120, or combinations thereof) may be formed of a transparent material. In this manner, interior components of autoinjector 100 may function as visually-identifiable features 130 for reflecting a state and/or indicia of autoinjector 100. For example, a user may observe the state and/or position of various components of autoinjector 100, such as plunger 220, syringe 210 with the medicament, and/or a needle to determine, e.g., whether autoinjector 100 has been previously actuated, damaged, tampered with and/or contains enough medicament for a subsequent injection.

Referring back to FIG. 7, electronic recordation device 710 is configured to capture image 715 and produce digital image data 720, and may comprise various hardware and/or software components for doing so, such as stand alone hardware and/or software components or hardware and/or software components situated within a smartphone, cell phone, personal digital assistant (PDA), tablet computer, laptop computer, desktop computer, webcam, electronic camera, or the like. Electronic recordation device 710 is also configured to transmit digital information, which may include image data 720, using one or more of various communication mediums, such as a wireless channel and/or a wired connection. Exemplary electronic recordation devices 710 applicable to various embodiments of the present invention are disclosed, for example, in U.S. Pat. No. 9,223,932, the entire disclosure of which is incorporated herein by reference and for all purposes.

In one embodiment, electronic recordation device 710 includes photograph capturing software operable to continuously analyze a viewing area until a target object is recognized, at which point electronic recordation device 710 captures image 715 automatically. The target object may include, for example, visually identifiable feature 130 of autoinjector 100. In an alternative embodiment, the photograph capturing software visually and/or audibly directs a user to properly position the target object. For example, the photograph capturing software may visually and/or audibly direct the user to properly position visually identifiable feature 130 of autoinjector 100 within a viewing area to capture image 715 therefrom. Exemplary automatic image capture and positioning systems/software are disclosed in U.S. Pat. Nos. 8,322,622 and 8,532,419, the entire disclosures of which are incorporated herein by reference and for all purposes.

Image data 720 generated by electronic recordation device 710 may also include, for example, metadata associated with the capture of image 715 or transmission of image data 720, such as, for example, a date, a time, an Internet Protocol address, a location (e.g., via GPS), or the like. Moreover, image data 720 may include input data provided by a user via an input device, such as a keyboard, mouse, touchscreen, or the like (not shown), including patient identifying information, date and time of last dosage, other medications administered, recent meals eaten, weight, blood pressure, vital signs, dosing history with an autoinjector, an anticipated time for a future dose, etc.

As described above with respect to FIG. 7, computing system 725 is operable to process image data 720 generated by electronic recordation device 710 into interpreted data to be provided to a stakeholder, such as, for example, a patient using the autoinjector, a doctor, an insurer, a caregiver, and/or a pharmaceutical company. The interpreted data may include encrypted or unencrypted data, and may be provided to the stakeholder by being saved on an accessible hard drive (such as, e.g., via a database entry or electronic medical record), displayed on a screen, or transmitted electronically. In the event the stakeholder is an individual such as, for example, the patient, caregiver or doctor, the interpreted data may be delivered directly by e-mail, text message (MMS or SMS), pager signal, mobile application messaging systems, or by other electronic transfer methods. For example, in one embodiment, the interpreted data is sent via a text message and includes instructions to be followed subsequent to a patient receiving a dose of medicament from autoinjector 100. In the event the stakeholder is part of the medical community, such as, for example, a doctor, hospital, insurer, pharmaceutical company, or researcher, the interpreted data may be used to track patient compliance with a treatment plan and/or to determine efficacy of treatment. For this purpose, the stakeholder may provide the patient with a computer executable software application operable to communicate various information to the patent, such as, for example, personalized messages, a treatment history, a treatment plan, suggestions for improving compliance with the treatment plan, diagnoses, treatment modifications or adjustments, assistance with proper use of autoinjector 100, information regarding product recalls, or combinations thereof.

The interpreted data may have patient-identifying information stripped therefrom and/or be saved, stored and/or transmitted in accordance with privacy laws such as The Health Insurance Portability and Accountability Act of 1996 (HIPAA). The interpreted data may also include other information linked thereto, such as, for example, information indicative of the medical history of the patient including allergies and other prescriptions, follow-up instructions and warnings associated with the medicament delivered by autoinjector 100, metadata associated with image data 720, links or videos with additional information, and/or compiled data, such as usage of medicament over time, use of a medicine lot by multiple patients, usage of a type of autoinjector 100, and combinations thereof.

Examples of desired stakeholders and the types of interpreted data they may desire are disclosed in U.S. Pat. No. 8,226,610 and “Development of Smart Injection Devices: Insights from the Ypsomate® Smart Case Study,” Schneider, Dr. Andreas: On Drug Delivery Feb. 10, 2016 at 6, the entire disclosures of which are incorporated by reference herein for all purposes.

The image processing performed by computing system 725 to produce the interpreted data may include, for example, a process by which image 715 obtained from visually identifiable feature 130 of autoinjector 100 is detected, recognized, identified, and/or interpreted via image analysis techniques. Such techniques may include, for example, Optical Character Recognition (“OCR”), visual recognition systems, such as those used to detect and recognize license plates, barcode and QR code readers, machine learning techniques, and the like. Exemplary image analysis and related systems applicable to the present invention are disclosed in the following references: U.S. Pat. No. 7,069,240; U.S. Patent Application Publication No. 2011/0183712; Ondrej Martinsky “Algorithmic and mathematical principles of automatic number plate recognition systems,” Brno University of Technology, 2007. Retrieved 2016-04-27; and Oskar Linde and Tony Lindeberg “Composed Complex-Cue Histograms: An Investigation of the Information Content in Receptive Field Based Image Descriptors for Object Recognition,” Computer Vision and Image Understanding 116: 538-560, 2012; the entire disclosures of which are incorporated herein by reference and for all purposes.

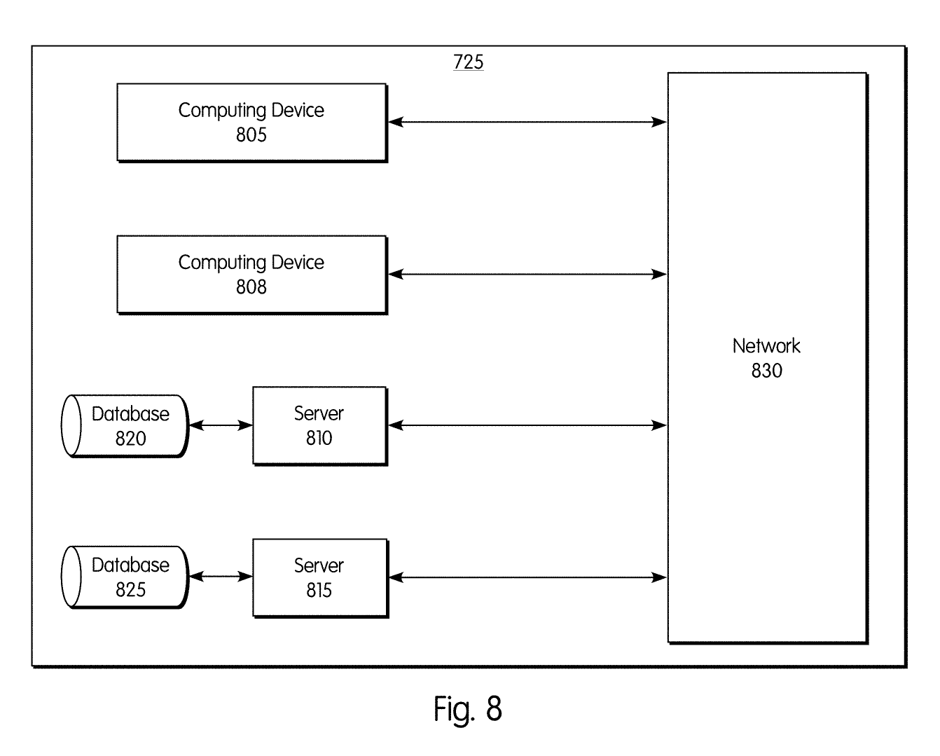

Referring now to FIG. 8, there is seen an exemplary computing system 725 in accordance with the present invention for generating and providing data interpreted from image data 720 supplied by electronic recordation device 710. Computing system 725 is but one example of a suitable computing environment and is not intended to suggest any limitation as to the scope of use or functionality thereof. Other general or special purpose computing system environments or configurations may be used. Examples of well-known computing systems, environments, and/or configurations that may be suitable for use include, but are not limited to, personal computers (“PCs”), server computers, handheld or laptop devices, multi-processor systems, microprocessor-based systems, network PCs, minicomputers, mainframe computers, cell phones, tablets, embedded systems, distributed computing environments that include any of the above systems or devices, and the like.

In the depicted embodiment, exemplary computing system 725 includes, inter alia, one or more computing devices 805, 808 and one or more servers 810, 815 with corresponding databases 820, 825 inter-connected via network 830. Network 830 may include any appropriate network, such as a wired or wireless network, that permits electronic communication among computing devices 805, 808 and servers 810, 815, and may include an external network, such as the Internet or the like, and/or a direct or indirect coupling to an external network.

Although FIG. 8 depicts computing devices 805, 808 located in close proximity to servers 810, 815, this depiction is exemplary only and not intended to be restrictive. For example, with respect to embodiments in which network 830 includes the

Internet, computing devices 805, 808 may be respectively positioned at any physical location. Also, although FIG. 8 depicts computing devices 805, 808 coupled to servers 810, 815 via network 830, computing devices 805, 808 may be coupled directly to servers 810, 815 via any other compatible network including, without limitation, an intranet, local area network, or the like.

Exemplary computing system 725 may use a standard client server technology architecture, which allows users of system 725 to access information stored in databases 820, 825 via custom user interfaces. In some embodiments of the present invention, the processes are hosted on one or more external servers accessible via the Internet. For example, in one embodiment, users can access exemplary computing system 725 using any web-enabled device equipped with a web browser. Communication between software components and sub-systems may be achieved by a combination of direct function calls, publish and subscribe mechanisms, stored procedures, and/or direct SQL queries; however, alternate components, methods, and/or sub-systems may be substituted without departing from the scope of the invention. Also, alternate embodiments are envisioned in which computing devices 805, 808 access one or more external servers directly via a private network rather than via the Internet.

In one embodiment, computing devices 805, 808 interact with servers 810, 815 via HyperText Transfer Protocol (“HTTP”). HTTP functions as a request-response protocol in client-server computing. For example, a web browser operating on computing device 805 may execute a client application that allows it to interact with applications executed by one or more of servers 810, 815. The client application submits HTTP request messages to the servers 810, 815, which provide resources such as HTML files and other data or content, or perform other functions on behalf of the client application. The response typically contains completion status information about the request as well as the requested content. However, alternate methods of computing device/server communications may be substituted without departing from the scope of the invention, including those that do not utilize HTTP for communications.

The number of servers 810, 815 and databases 820, 825 are merely exemplary and others may be omitted or added without departing from the scope of the present invention. Further, databases 820, 825 may be combined into a single database and/or be included in respective servers 810, 815. It should also be appreciated that one or more databases, including databases 820, 825 may be combined, provided in or distributed across one or more of computing devices 805, 808, dispensing with the need for servers 810, 815 altogether.

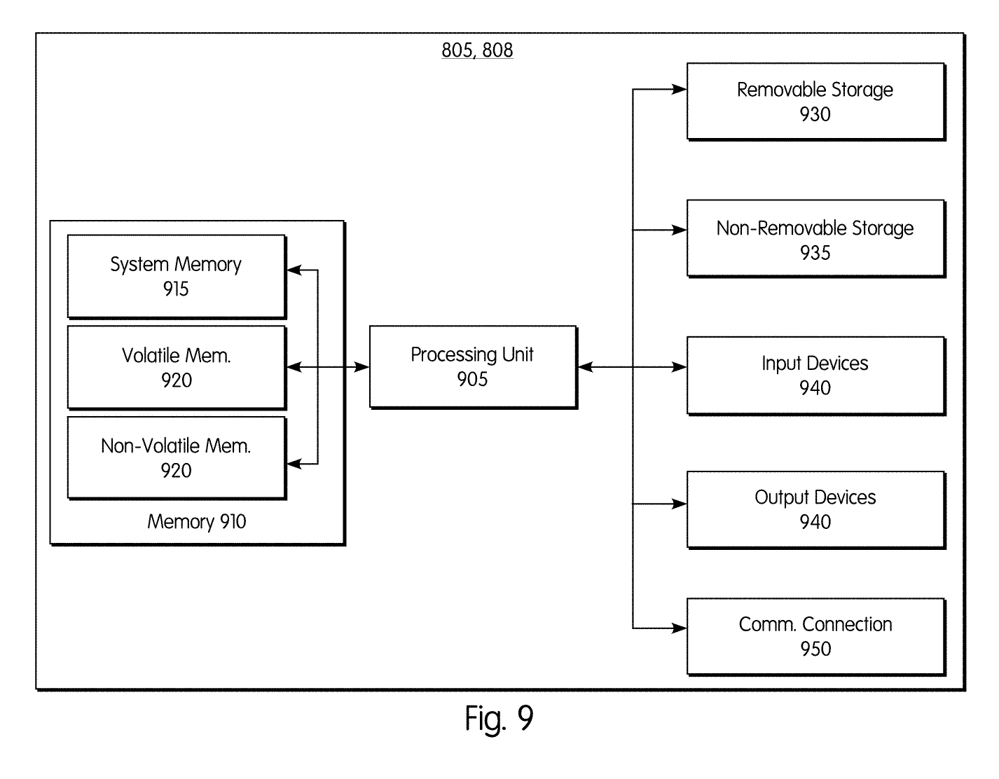

In its most basic configuration, as depicted in FIG. 9, each of computing devices 805, 808 includes at least one processing unit 905 and at least one memory 910. Depending on the exact configuration and type of computing devices 805, 808, memory 910 may include, for example, system memory 915, volatile memory 920 (such as random access memory (“RAM”)) non-volatile memory 925 (such as read-only memory (“ROM”), flash memory, etc.), and/or any combination thereof. Additionally, computing devices 805, 808 may include any web-enabled handheld device (e.g., cell phone, smart phone, or the like) or personal computer including those operating via Android™, Apple®, and/or Windows® mobile or non-mobile operating systems.

Computing devices 805, 808 may have additional features/functionality. For example, as shown in FIG. 9, computing devices 805, 808 may include removable and/or non-removable storage 930, 935 including, but not limited to, magnetic or optical disks or tape, thumb drives, and/or external hard drives as applicable. Computing devices 805, 808 may also include input device(s) 940 such as a keyboard, mouse, pen, voice input device, touch input device, etc., for receiving input from a user, as well as output device(s) 945, such as a display, speakers, printer, etc.

Computing devices 805, 808 may also include communications connection 950 to permit communication of information with other devices, for example, via a modulated data signal (such as a carrier wave or other transport mechanism); i.e., a signal that includes one or more characteristics that are changed in accordance with the information to be transmitted. Transmission of the information may be accomplished via a hard-wired connection or, alternatively, via a wireless medium, such as a radio-frequency (“RF”) or infrared (“IR”) medium.

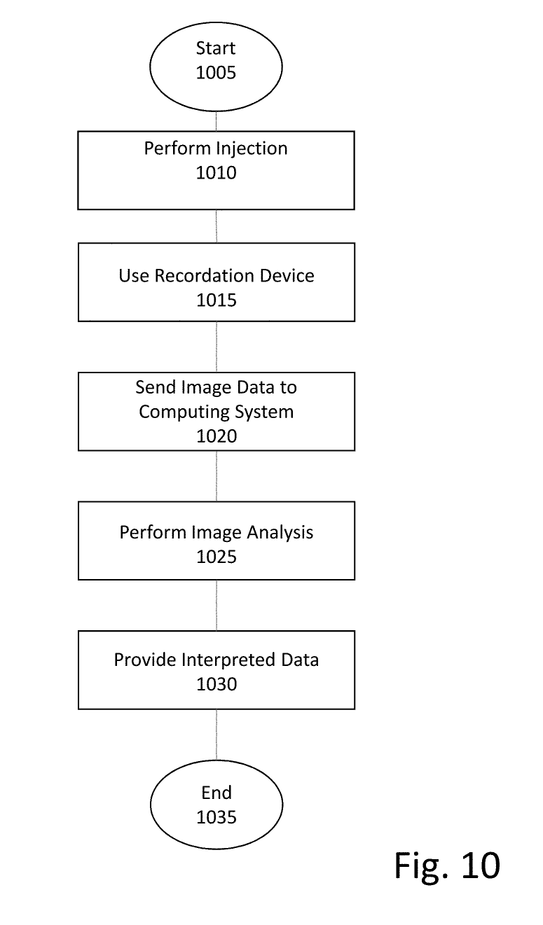

Referring now to FIG. 10, there is seen an exemplary flow chart depicting a process for administering a medicament and monitoring a medical device, in accordance with the present invention. The process begins at step 1005 and proceeds to step 1010, at which a patient or caregiver administers medicament to the patient using autoinjector 100. Then, at step 1015, the patient or caregiver uses electronic recordation device 710 to capture image 715 of visually-identifiable feature(s) 130 indicative of a state and/or indicia of autoinjector 100. Electronic recordation device 710 processes image 715 to generate image data 720, which is then communicated to exemplary computing system 725 at step 1020. The process proceeds to step 1025, at which computing system 725 processes image data 720 to produce interpreted data indicative of the state and/or indicia of autoinjector 100. The interpreted data is then provided to a stakeholder at step 1030, and the process ends at step 1035.

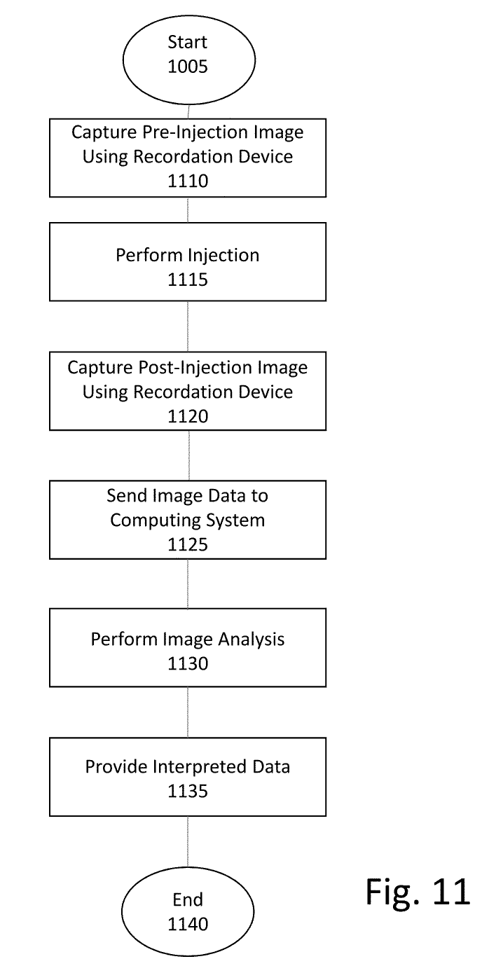

Referring now to FIG. 11, there is seen an exemplary flow chart depicting another process for administering a medicament and monitoring a medical device, in accordance with the present invention. The process begins at step 1105 and proceeds to step 1110, at which a patient or caregiver uses electronic recordation device 710 to capture a pre-injection image 715a of visually-identifiable feature(s) 130 indicative of a state and/or indicia of autoinjector 100. After pre-injection image 715a is acquired, the process proceeds to step 1115. At this step, the patient or caregiver administers medicament to the patient using autoinjector 100. The process proceeds to step 1120, at which the patient or caregiver uses electronic recordation device 710 to capture a post-injection image 715b of visually-identifiable feature(s) 130 of autoinjector 100. Electronic recordation device 710 processes pre-injection and post-injection images 715a, 715b to generate image data 720, which is then communicated to exemplary computing system 725 at step 1125. The process proceeds to step 1130, at which computing system 725 processes image data 720 to produce interpreted data indicative of the state and/or indicia of autoinjector 100. In the present example, image data 720 is processed to produce interpreted data indicative of a change in a state of autoinjector 100, for example, a change in an amount medicament within autoinjector 100. The interpreted data is then provided to a stakeholder at step 1135, and the process ends at step 1140.

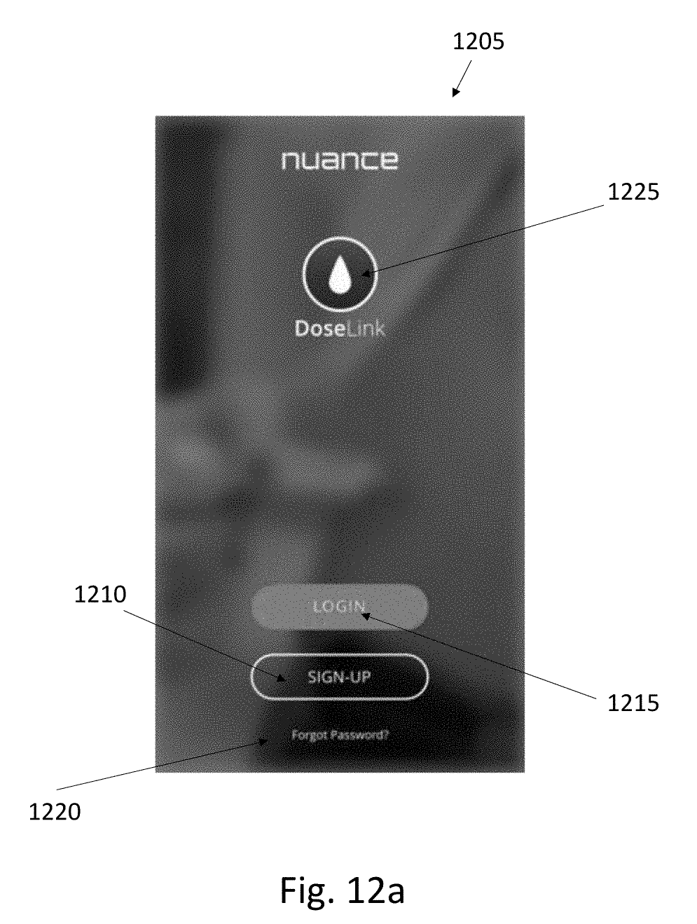

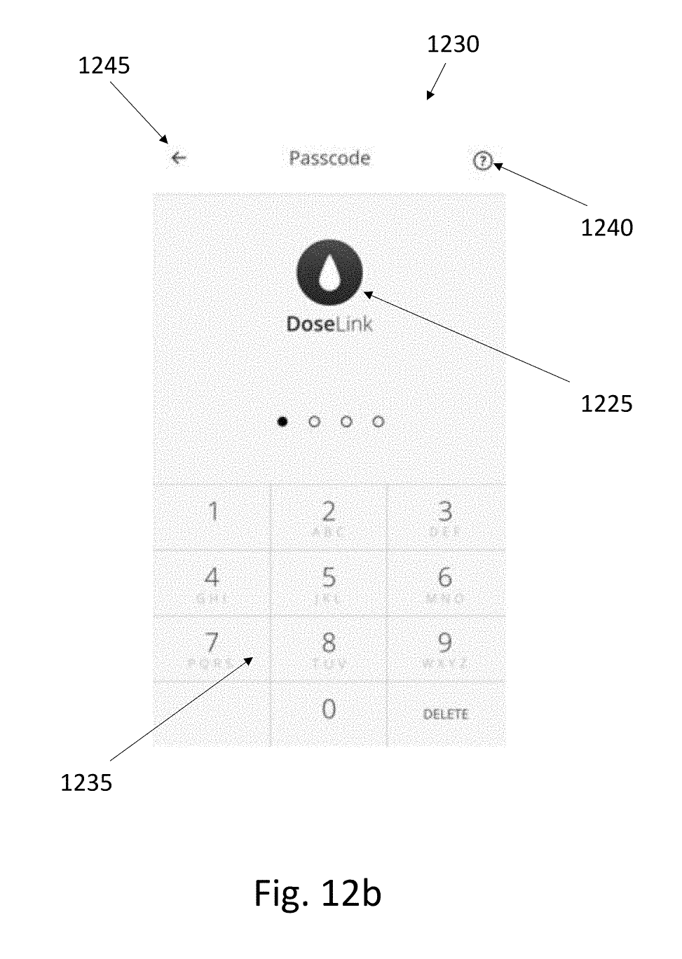

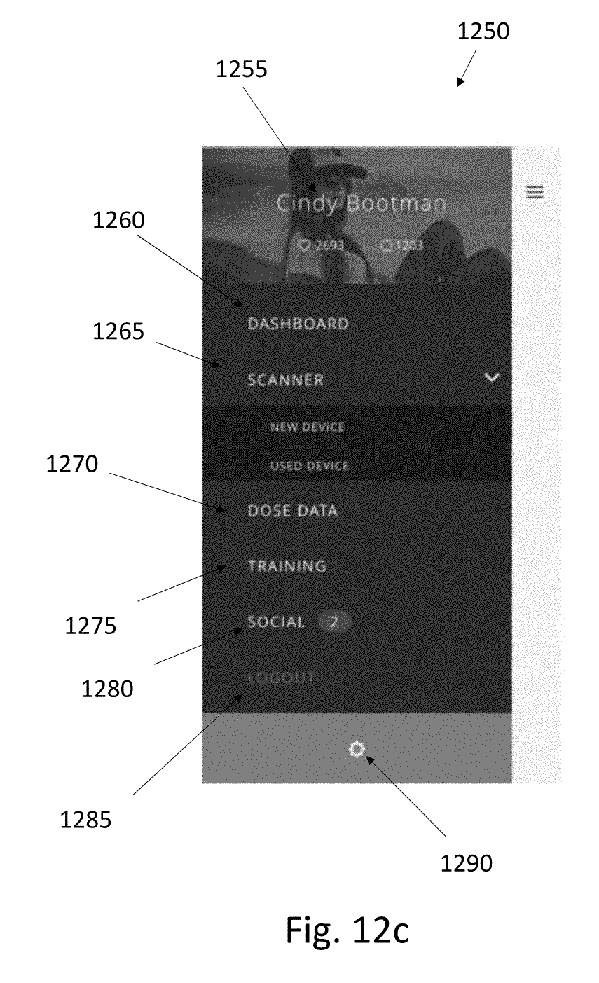

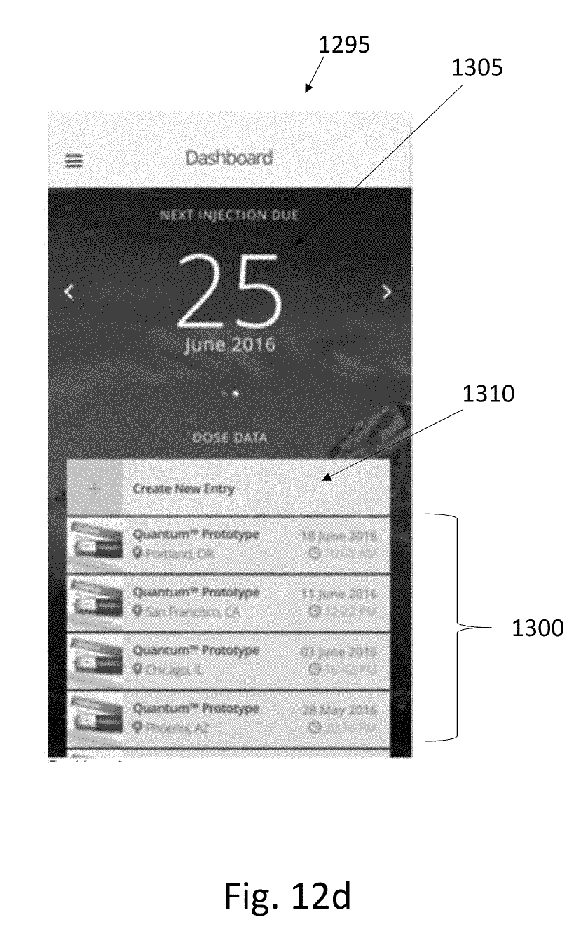

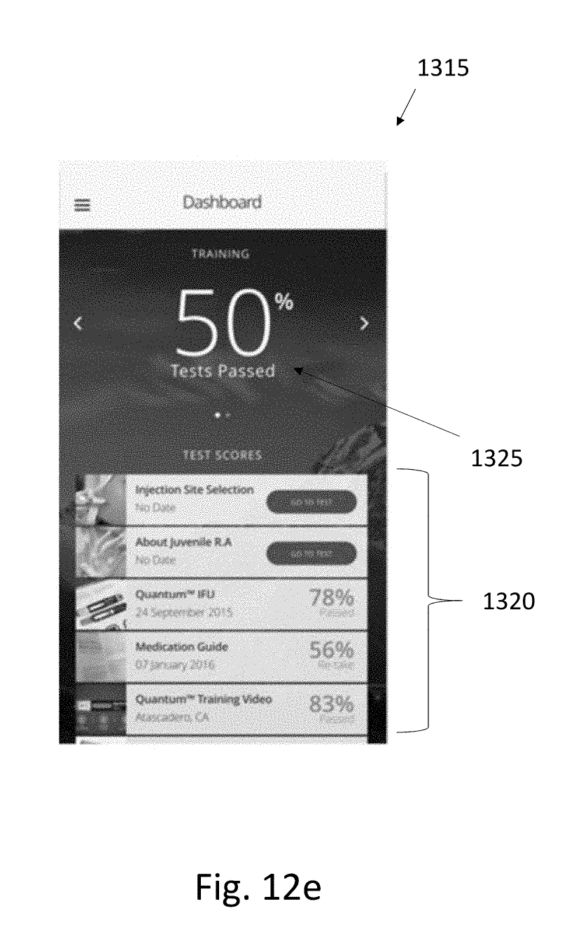

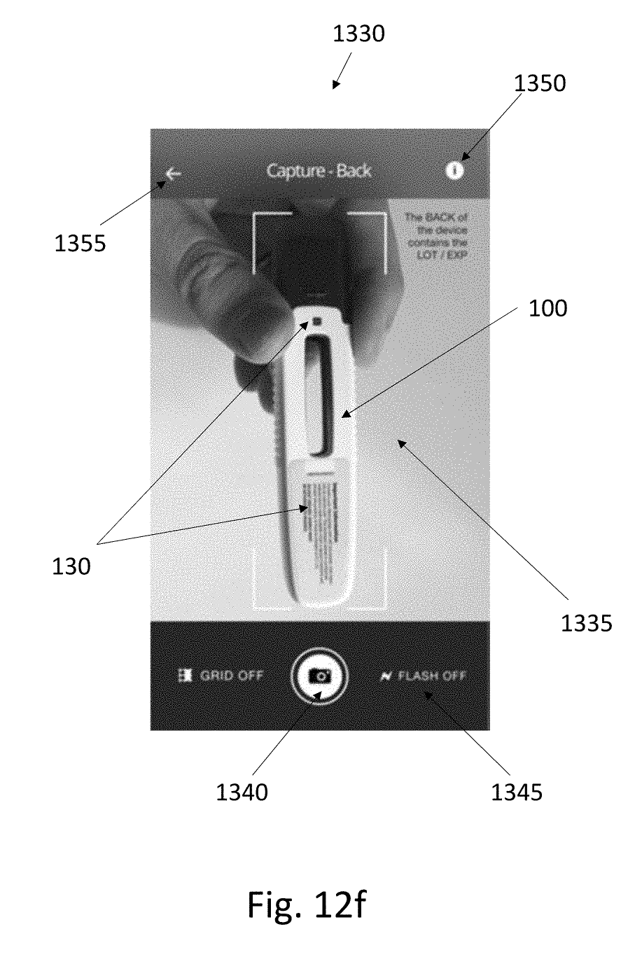

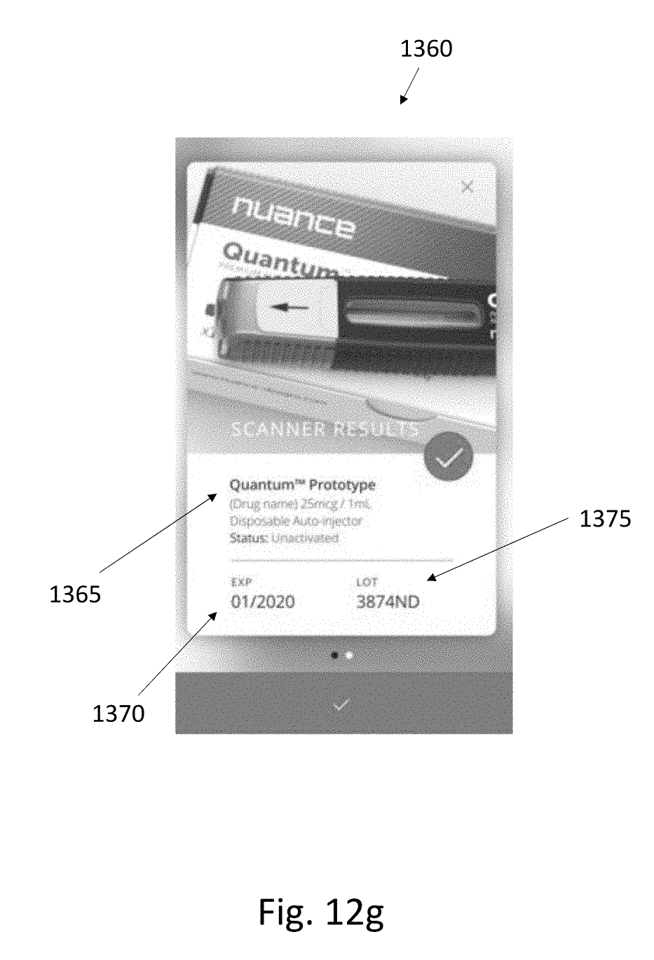

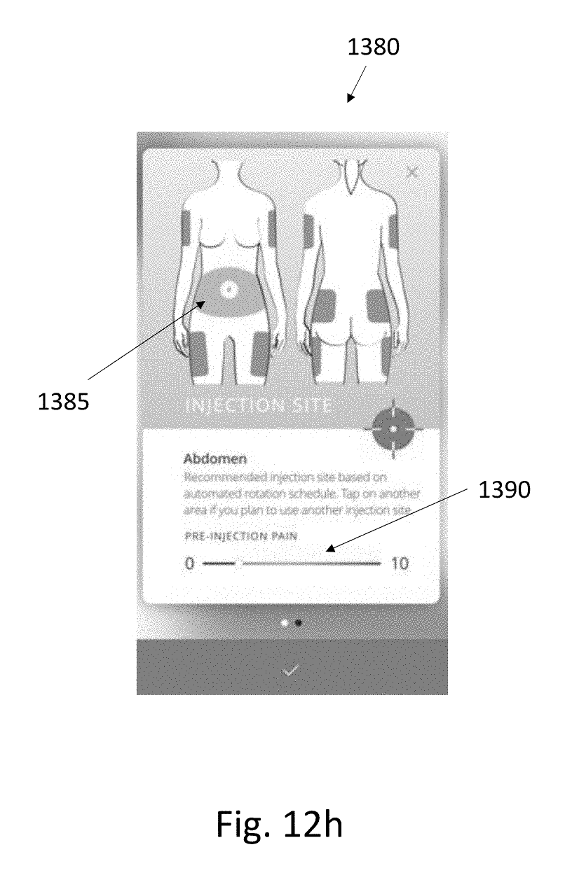

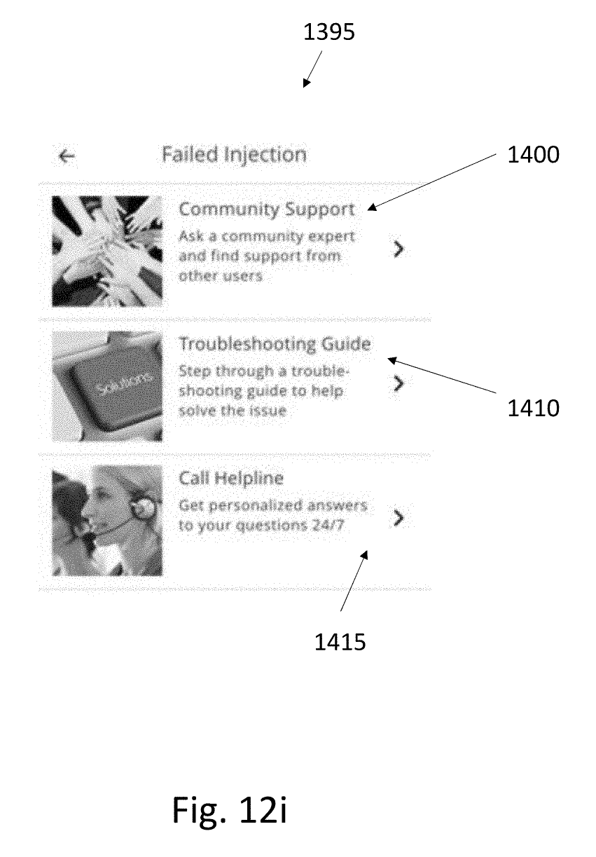

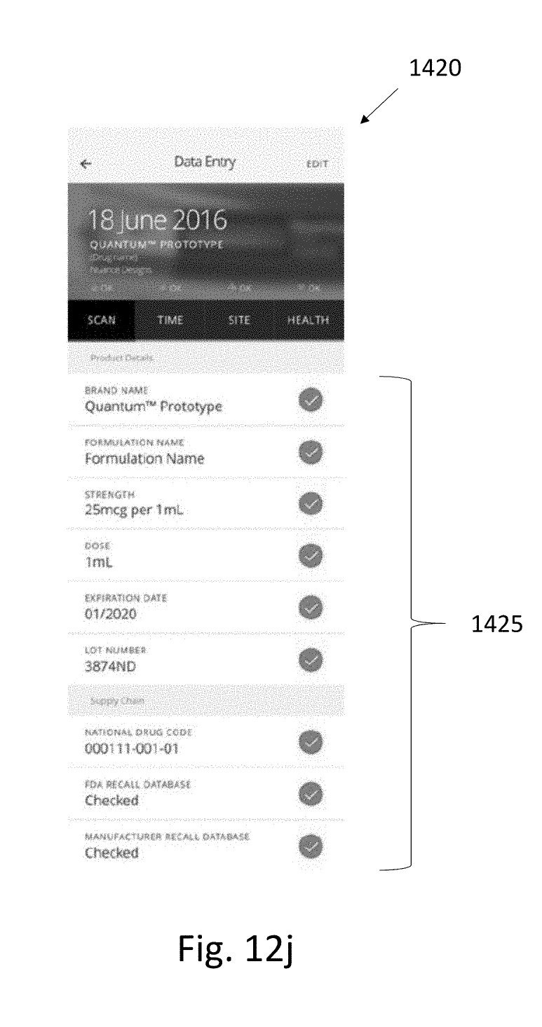

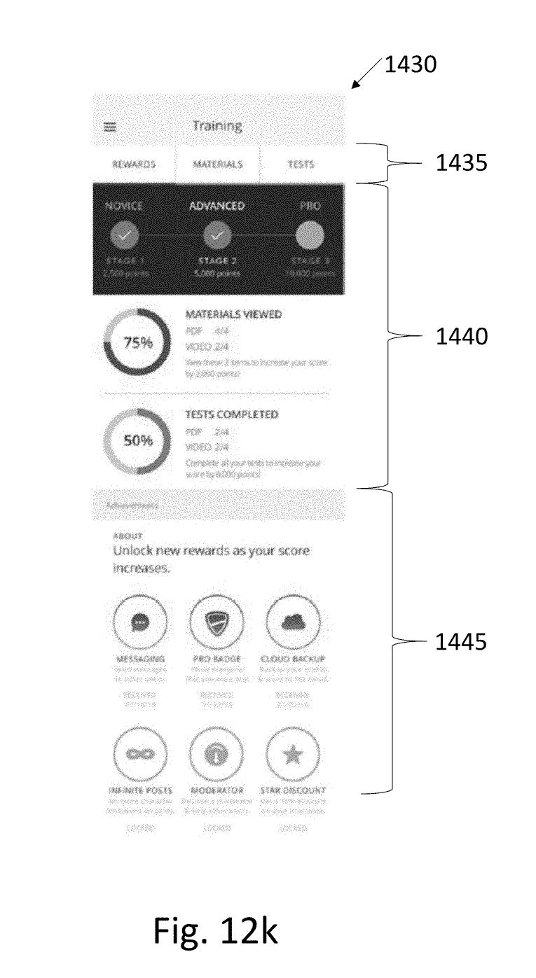

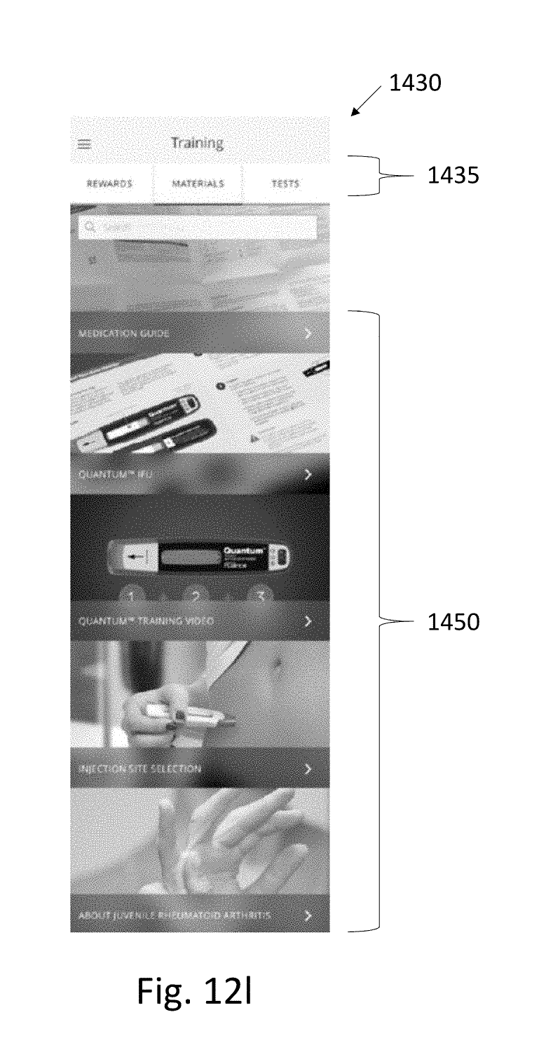

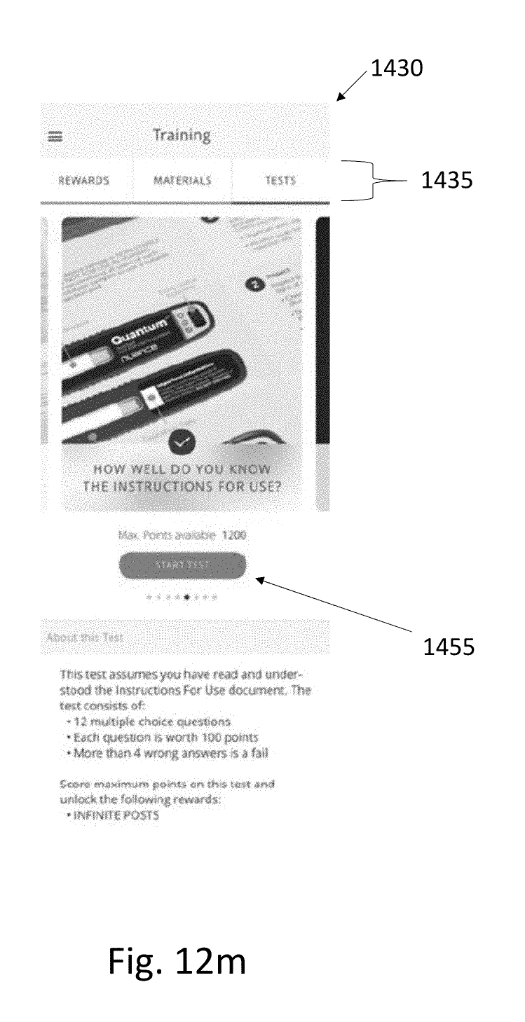

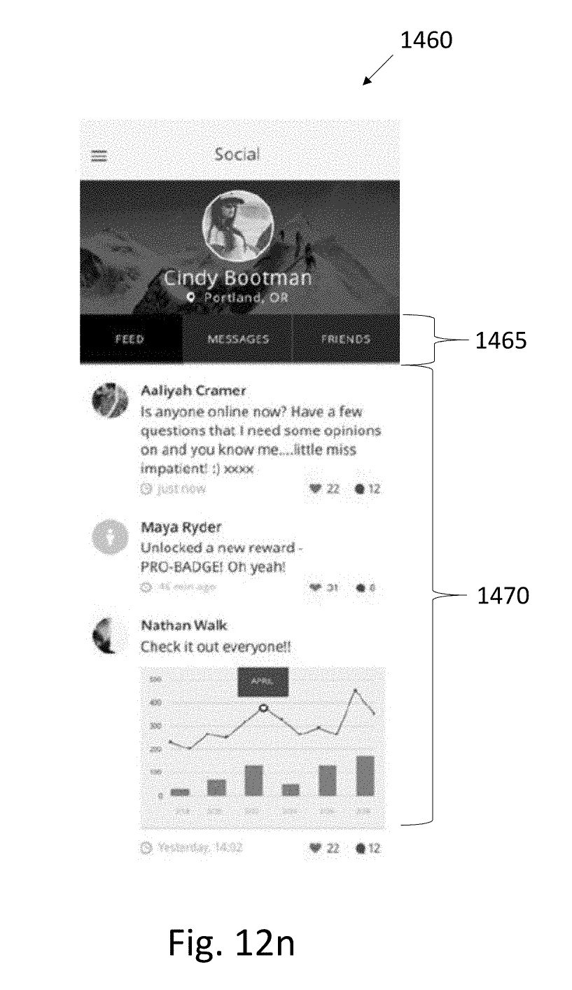

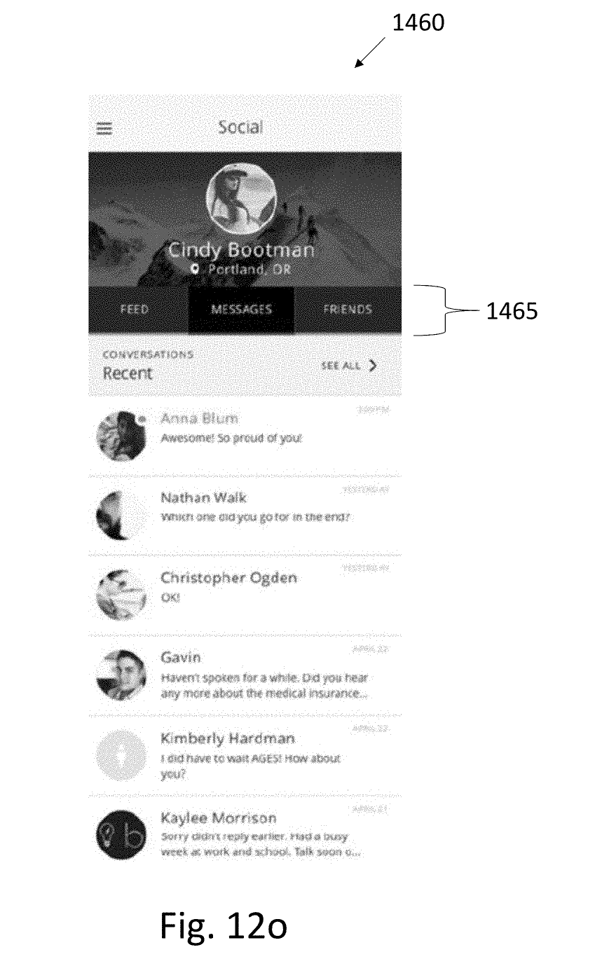

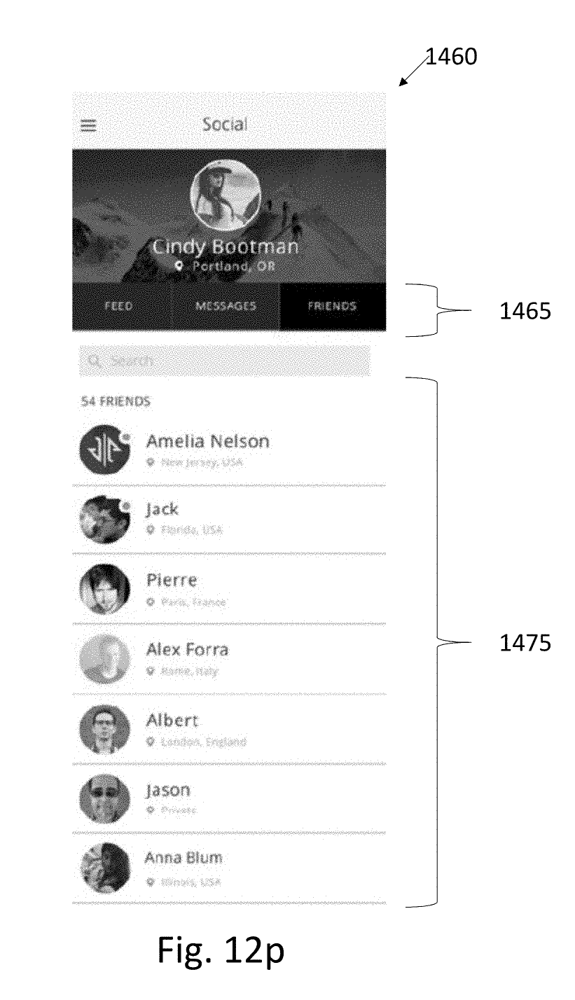

As described above, electronic recordation device 710 and computing system 725 may comprise various hardware and/or software components configured to capture image 715 and produce image data 720 digitally. Referring now to FIGS. 12a through 12p, there is seen various exemplary screen shots of an inventive medical device monitoring system in the form of a mobile application to be executed, for example, on a smartphone or tablet of a user, such as a patient or caregiver. The mobile application is configured to communicate with a stakeholder over the Internet.

Upon launching the application, the user is presented with an opening screen 1205, such as the one shown in FIG. 12a. Opening screen 1205 provides the user with a sign-up option 1210 that permits him/her to set up an account with the stakeholder, such as, for example, a medical establishment, doctor, insurance company or the like. When setting up the account, the user selects an available user name and password, which he/she may then use to access the account via a login option 1215. In the event the user forgets or misplaces his/her password, a “Forgot Password” option 1220 provides a means by which the user may retrieve and/or reset his/her password or other account credentials upon completion of an appropriate authentication protocol. Opening screen 1205 may also display a logo, marketing or other information, such as, for example, corporate logo 1225 associated with the stakeholder.

In one embodiment, the password comprises a numerical code, such as a four or six digit alphanumeric code, which may be entered by the user, such as via the Passcode screen 1230 depicted in FIG. 12b. Passcode screen 1230 includes a graphical keypad 1235, by which the user may enter the code for accessing the application. In addition to or in lieu of providing the code, access to the application may be authenticated via fingerprint, face recognition, iris recognition, or other biometric technology, such as that provided on various Apple® and Android (e.g., Samsung) mobile devices. In the event the user experiences difficulty logging into the application, a “help” option 1240 may be selected for accessing information that may assist the user. A “back” option 1245 is also provided for returning to opening screen 1205.

After the user is properly authenticated, the application presents a main menu of options to the user, such as via Main Menu screen 1250 depicted in FIG. 12c. Main Menu screen 1250 displays information specific to the account of the user, such as, for example, name information 1255 associated with the patient or caregiver and/or other personal information and details. Main Menu screen 1250 also displays one or more user options associated with various functions of the application, such as a Dashboard option 1260, a Scanner option 1265 (with “New Device” and “Used Device” sub-options), a Dose Data Option 1270, a Training option 1275, a Social option 1280, a Logout option 1285 and a Settings option 1290.

Upon selecting the Dashboard option 1260, the user is presented with a dashboard screen, such as Dashboard screen 1295 depicted in FIG. 12d. Dashboard screen 1295 displays information associated with a medical history of the patient, such as, for example, the patient's injection history 1300, and a due date 1305 or other reminder for informing the user of timing information associated with a subsequent injection. Entries into the patient's injection history 1300 may be recorded automatically by the application (see below) or be entered manually via a “Create New Entry” option 1310, which provides the user with various prompts by which information associated with a medical event, such as an injection, may be inputted into and recorded by the application. Additional information may be displayed to the user by navigating (or swiping) across Dashboard screen 1295. For example, in one embodiment, swiping across Dashboard screen 1295 causes the Training Status Screen 1315 of FIG. 12e to be displayed. Training Status Screen 1315 displays various status information 1320 associated with the user's progress with various training materials or courses (such as training videos), as well as other statistical information 1325 associated with the user's training.

Scanner option 1265 may be selected by the user to perform pre and post-injection scans of a medical device, such as autoinjector 100. Upon selecting Scanner option 1265 (see Main Menu screen 1250 depicted in FIG. 12c), the user selects either the “New Device” sub-option or the “Used Device” sub-option depending upon whether he/she intends to perform an injection of medicament using a new or used autoinjector 100.

After selecting either the “New Device” or “Used Device” sub-option, the application presents the user with Scanning screen 1330 depicted in FIG. 12f. When presenting Scanning screen 1330, the application accesses and displays a viewing area 1335 from an on-board camera of the mobile device running the application. The user positions autoinjector 100 within viewing area 1335 and depresses the Snapshot button 1340 to take a picture, thereby capturing a pre-injection image 715 of visually-identifiable features 130 of autoinjector 100. In another embodiment, the application automatically takes the picture upon detection of proper alignment of autoinjector 100 within viewing area 1335. In the embodiment depicted in FIG. 12f, visually-identifiable features 130 of autoinjector 100 are positioned on the front and back thereof and include features indicative of a state and/or indicia of autoinjector 100, such as, for example, a product name, lot number, amount of medicament, and/or expiration date. In the event extra lighting is needed to illuminate autoinjector 100 before the scan, the user may select a Flash option 1345. The user may also select an Information option 1350 for additional information concerning the process for scanning or a Back button 1355 to return to Main Menu screen 1250 depicted in FIG. 12c.

After the scan is complete, the application performs various checks, such as, for example, confirming that the name of the drug scanned matches an associated prescription, whether autoinjector 100 is new or used, whether the time and date of the imminent injection correlates to the prescription and the last recorded injection, and/or whether a scanned lot number is listed on any recall databases. In another embodiment, the application authenticates autoinjector 100 with an associated pharmaceutical company or other organization, such as via appropriate communication over the Internet, to detect possible market diversion of autoinjector 100 and/or to ensure that autoinjector 100 is not counterfeit. The application then presents the results of the scan via Scanner Results screen 1360 depicted in FIG. 12g. In the embodiment depicted in FIG. 12g, Scanner Results screen 1360 presents Drug Information 1365 (such as the brand name and type of medicament, as well as the new/used status of autoinjector 100), Expiration Date 1370 and Lot Number 1375 associated with autoinjector 100 and/or a medicament contained therein.

After Scanner Results screen 1360 is presented to the user, the application displays Injection Site screen 1380 depicted in FIG. 12h. Injection Site screen 1380 presents a graphical depiction of a human body with various injection sites and highlights a Recommended Site 1385 based on a rotation schedule of injections assigned to the patient. The user may accept the Recommended Site 1385 or, alternatively, highlight an alternative site for the imminent injection. Injection Site screen 1380 also presents a Pre-Injection Pain option 1390, which allows the user to record a level of pain at the injection site prior to the injection, for example, by selecting a level of pain from zero to ten.

After the user records the selected injection site and associated pre-injection pain level, the application instructs the user to perform the injection. In one embodiment, access to training materials (such as e-books or videos) is provided at this step in the event the user wishes to view a step-by-step guide on how to perform the injection correctly. If the injection was successful, the user indicates as such and the application returns to Scanning screen 1330 depicted in FIG. 12f, at which the user performs a post-injection scan of visually-identifiable features 130 of autoinjector 100. After completion of the post-injection scan, the application compares the image data 720 of the pre-injection and post-injection scans to determine whether the injection successfully administered a correct amount of medicament. The user is also presented with an option to select a level of post-injection pain at the injection site.

After the user selects the level of post-injection pain at the injection site, the application records various information associated with the injection. In one embodiment, the application presents a Data Record screen 1420 (see FIG. 12j) displaying various Captured and Other Information 1425 from the pre and post-injection scans, including, for example, the brand name of autoinjector 100 or a medicament contained therein, a formulation name, a formulation strength, a dose, an expiration date, a lot number, a national drug code, results of an FDA recall database check, results of a manufacturer recall database check, the site of the injection, levels of pre and post-injection pain indicated by the user, and/or the date, time and geographic location of the injection. In another embodiment, the application obtains and records additional health related information received from other health related applications installed on the mobile device and/or external health monitoring devices (such as an Apple i-Watch®, FitBit® monitor or the like), such as, for example, the patient's weight, heart rate, blood pressure, calorie intake, calorie burn rate, blood glucose level, etc.

If the injection was unsuccessful, the user indicates as such, after which the application presents various options for assistance. For example, in one embodiment, the application displays a Troubleshooting screen 1395 (see FIG. 12i) which presents a Community Support option 1400, a Troubleshooting Guide option 1405 and a Helpline option 1410. Community Support option 1400 allows the user to contact other users and experts online, such as by viewing and participating in a support forum, instant messaging or the like. Troubleshooting Guide option 1405 provides information and other resources, such as step-by-step guides, to assist the user in solving various issues. Helpline option 1410 allows the user to speak with an assistant or other expert over the phone or by instant message in order to troubleshoot various problems associated with failed injections and other issues.

Referring back to Main Menu screen 1250 depicted in FIG. 12c, selection of Training option 1275 causes the application to present various options for viewing training materials associated with the user's treatment plan. In one embodiment, the application displays Training Screen 1430 depicted in FIG. 12k. Training Screen 1430 includes sub-menus 1435 (“Rewards,” “Materials,” and “Tests” sub-menus are displayed in FIG. 12k), a Progress Status Display 1440 for displaying various status messages associated with the user's progress with training, such as an indication of whether the user's current level of proficiency is “Novice,” “Advanced” or “Pro” depending on the amount of training materials already consumed by the user, an indication of a percentage of training materials already consumed, and/or a percentage of various tests already completed.

With the “Rewards” sub-menu 1435 selected, Training Screen 1430 also displays various Rewards 1445 available to the user based on the amount of training materials he/she has consumed, the score(s) of various written tests he/she took, and/or other factors, such as, for example, a reward allowing the user to message other users, a badge or other icon informing others of the user's proficiency with various training materials, a reward that permits the user to backup his/her account and record other information to an internet Cloud account, a reward that permits the user to author a certain amount of posts (such as an infinite amount of posts) on various messaging boards, chat rooms or forums, a reward that bestows on the user a “Moderator” status that permits him/her to moderate various chat rooms, messaging boards or forums associated with the application, and/or a reward that offers the user various discounts on products, such as discounts on medically-related products.

With the “Materials” sub-menu 1435 selected, Training Screen 1430 displays various options by which the user may select training materials 1450 (such as informational videos) to view and consume (see FIG. 12l). In one embodiment, the application is operable to measure user interaction with training materials 1450, such as, for example, by analyzing scroll behavior, time spent by the user with various training materials 1450, whether the user skips certain sections of training materials 1450, whether the user repeats viewing of certain sections of training materials 1450, and/or the frequency with which the user views training materials 1450. This information may be used to assign the user a score, by which the user can track his/her progress and proficiency with training materials 1450. With the “Tests” sub-menu 1435 of Training Screen 1430 selected, the user is presented with the option 1455 to take various online written tests via the application (see FIG. 12m). Depending on the results of these tests, the user's score may increase, decrease or remain unchanged. As described above, progressively higher scores may unlock various rewards available to the user.

Referring back to Main Menu screen 1250 depicted in FIG. 12c, selection of Social option 1280 causes the application to display a Social screen 1460 (see FIG. 12n) permitting the user to engage with other users of the application. Social screen 1460 includes sub-menus 1465 (“Feed,” “Messages,” and “Friends” sub-menus are displayed in FIG. 12n). With the “Feed” sub-menu 1465 selected, the user is presented with a feed of messages and updates 1470 generated by other users. Users can create a social profile and access the feed from connected friendships or trending (or followed) topics of interest. With the “Messages” sub-menu 1465 selected, the user is presented with a screen permitting him/her to message and communicate with friends, other patients, doctors, healthcare providers, etc. from within the application (see FIG. 12o). With the “Friends” sub-menu 1465 selected, the user is presented with a screen permitting him/her to add or delete various individuals or other users of the application from a list of friends 1475 (see FIG. 12p).

It will be appreciated by those skilled in the art that changes could be made to the embodiments described above without departing from the broad inventive concept thereof. For example, the computer may be part of the electronic recordation device or it may be part of a remote cloud server. It is to be understood, therefore, that this invention is not limited to the particular embodiment disclosed, but it is intended to cover modifications within the spirit and scope of the present invention as defined by the appended claims.

What is claimed is:

1. A drug delivery device monitoring system comprising:
a drug delivery device having a visually-identifiable feature reflecting a state or an indicia of the drug delivery device;
an electronic recordation device configured to capture an image of the visually-identifiable feature and generate image data therefrom; and
a computing system operable to perform image analysis on the image data to generate interpreted data therefrom.

2. The system of claim 1, wherein the visually-identifiable feature includes a barcode, a QR code, a graduation line, a light-emitting diode, printed text, a holographic print, a microprint, infrared ink, ultraviolet ink, color-shifting ink, a watermark, a position, a display, a viewing window, an appearing mark, a disappearing mark or combinations thereof.

3. The system of claim 1, wherein the state of the drug delivery device includes an unused condition, a used condition, a time of use, a dosage volume, or combinations thereof.

4. The system of claim 1, wherein the indicia of the drug delivery device includes a lot number, an expiration date, instructions for use, a time of use, patient identifying information, or combinations thereof.

6. The system of claim 1, wherein the drug delivery device is an autoinjector.

7. The system of claim 1, wherein the interpreted data is provided to a stakeholder.

8. A method of monitoring a medical device, comprising:
performing an injection using a drug delivery device, the drug delivery device having a visually-identifiable feature reflecting a state or an indicia of the drug delivery device;
using a recordation device to capture an image of the visually-identifiable feature and generate image data therefrom;
using a computing system to perform image analysis on the image data to generate interpreted data therefrom.

9. The method of claim 8, wherein the step of using the recordation device to capture the image of the visually-identifiable feature and generate image data therefrom includes:
using the recordation device to capture a first image of the visually-identifiable feature before the injection; and
using the recordation device to capture a second image of the visually-identifiable feature after the injection;
wherein the recordation device generates the image data in accordance with the first and second images.

10. The method of claim 9, wherein the step of using the computing system to perform image analysis on the image data to generate interpreted data therefrom includes:
using the computing system to compare the image data obtained from the first and second images to determine a change in the state of the drug delivery device.

11. The method of claim 8, wherein the visually-identifiable feature includes a barcode, a QR code, a graduation line, a light-emitting diode, printed text, a holographic print, a microprint, infrared ink, ultraviolet ink, color-shifting ink, a watermark, a position, a display, a viewing window, an appearing mark, a disappearing mark or combinations thereof.

12. The method of claim 8, wherein the state of the drug delivery device includes an unused condition, a used condition, a time of use, a dosage volume, or combinations thereof.

13. The method of claim 8, wherein the indicia of the drug delivery device includes a lot number, an expiration date, instructions for use, a time of use, patient identifying information, or combinations thereof.

14. The method of claim 8, wherein the drug delivery device is an autoinjector.

15. The method of claim 8, wherein the interpreted data is provided to a stakeholder.

16. A non-transitory medium, comprising:
computer executable instructions for an application executable by a user on a mobile device with a camera, the instructions operable to perform the following steps:
scanning a drug delivery device using the camera of the mobile device to capture an image of a visually-identifiable feature reflecting a state or an indicia of the drug delivery device;
generating image data from the captured image;
processing the image data to generate interpreted data; and
recording the interpreted data.

17. The non-transitory medium of claim 16, wherein the computer executable instructions are further operable to display a training screen to the user, the training screen presenting training materials for consumption by the user.

18. The non-transitory medium of claim 17, wherein the training screen presents one or more options for taking a test based on the training materials.

19. The non-transitory medium of claim 18, wherein the computer executable instructions are further operable to provide the user with one or more rewards based on a score associated with the test.

20. The non-transitory medium of claim 16, wherein the computer executable instructions are further operable to display a social screen allowing the user to communicate with other users of the application.
